(19) United States
(12) Patent Application Publication
(10) Pub. No.: US 2013/0144138 A1
(43) Pub. Date: Jun. 6, 2013

(54) MEASURING DEVICE FOR GATHERING SIGNALS MEASURED IN VITAL TISSUE

(71) Applicants: Holger Jungmann, Gelsenkirchen (DE); Michael Schietzel, Herdecke (DE)

(72) Inventors: Holger Jungmann, Gelsenkirchen (DE); Michael Schietzel, Herdecke (DE)

(21) Appl. No.: 13/639,390

(22) PCT Filed: Apr. 11, 2011
(86) PCT No.: PCT/EP2011/001790

§ 371 Date: Jan. 16, 2013

(30) Foreign Application Priority Data
Apr. 9, 2010 (DE) 10 2010 014 592.0

(51) Int. Cl. A61B 5/1455

(52) U.S. Cl. CPC A61B 5/1455
USPC 600/322

(57) ABSTRACT

The invention relates to a measuring device comprising a light source device (Q1, Q2), a spectrometer device (1) and a measuring head structure (2), the measuring head structure being coupled with the light source device via a first optical waveguide (L1) and a second optical waveguide (L2) as well as with the spectrometer device via a third optical waveguide (L3), said optical waveguides leading to a contact surface provided by the measuring head structure. The outlet positions of the optical waveguides are adapted to each other such that the distances (a,b) of the outlet positions of the first and second optical waveguides are different from the outlet position of the third optical waveguide. In this manner, a measuring device is devised which is characterized in that it is highly insensitive to disturbing influences which due to the uneven scattering of the cell structures occur in vital tissue systems.

The invention concerns a measuring device for collecting test signals from living tissue, especially for determining the composition of body liquids as well as of maybe only temporarily vascular-bound substances.

Movable spectrometer are known, by which an analysis of temporarily vascular-bound substances can be done, by applying this spectrometer to a corresponding tissue area of a living being to be examined and by recording by this movable spectrometer the spectrum of reflected light emerging from the tissue. By means of the spectrum recorded in this way various substances present in the examined tissue area can be detected. These spectrometers can be structured as classic spectrometers, in which the incident light is split by optical means and the intensity of the split light is is measured by associating it to the wavelength. For avoiding movable parts the spectrometer can be formed in such a way that the light split according to its wavelength is led on a CCD array and is analyzed by it.

The object of the invention is to create solutions by which by means of a spectrometric measurement measured values can be generated that distinguish themselves by a particularly high representativity.

This task is solved according to the invention by a measuring device with a light source device, a spectrometer device, and a measuring head structure, wherein the measuring head structure is coupled with the light source device over a first light guide and a second light guide as well as with the spectrometer device over a third light guide, in which these light guides end in a support surface provided by the measuring head structure, and wherein the outlet positions of the light guides are coordinated in such a way that the distances of the outlet positions of the first and second light guide distinguish from the outlet position of the third light guide.

It is thus advantageously possible to create a measuring device that distinguishes itself by a high insensitivity to disturbances, which are present in living tissue systems due to the uneven scattering effect of cell structures.

Preferably the measuring head structure is configured in such a way, that the light guides enter substantially perpendicularly from behind into the support surface.

The distance of the outlet position of the first light guide from the outlet position of the third light guide is preferably greater than the distance of the outlet position of the second light guide from the outlet position of the third light guide.

The distance of the outlet position of the first light guide from the outlet position of the third light guide preferably corresponds to the distance of the outlet position of the first light guide from the outlet position of the second light guide.

The measuring head structure can be formed in such a way that the outlet positions of the light guide represent the vertices of a triangle, in which an interior angle defined between the legs extending towards the outlet position of the third light guide is in the range from 79° to 94°, preferably 89°.

A design of the measuring head structure that is particularly advantageous for measurements on vital human tissue is made in such a way that the distance of the outlet position of the first light guide from the outlet position of the third light guide amounts to 3.6 mm.

The distance of the outlet position of the second light guide from the outlet position of the third light guide preferably amounts to 2.3 mm.

The light source device according to a particular aspect of the present invention is configured in such a way that it comprises two separate LED light sources, which each are associated to one of the light guides. The light guides are preferably iron-free multifilaments.

Further particulars and characteristics of the invention result from the following description in connection with the drawing. The FIGURES show:

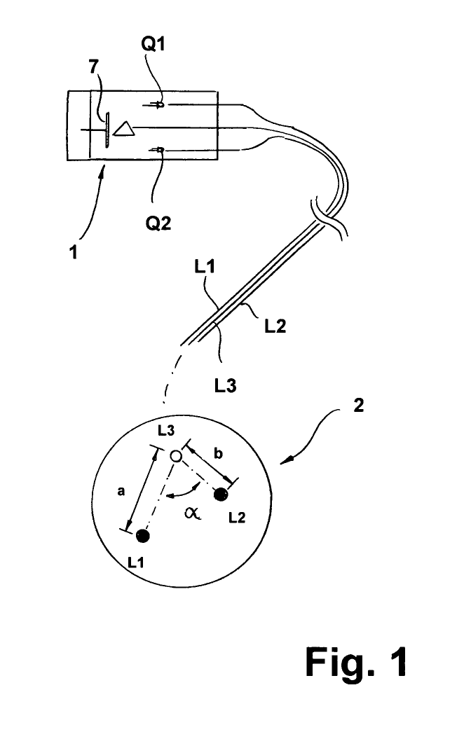

FIG. 1 a sketch to illustrate the structure of a mobile measuring device according to the invention.

FIG. 1 shows a movable measuring device with a light source device Q1, Q2, a spectrometer device 1 and a measuring head structure 2, the measuring head structure 2 being coupled with the light source device Q1, Q2 over a first light guide L1 and a second light guide L2 as well as with the spectrometer device 1 over a third light guide L3, these light guides 1, L2, L3 ending in a support surface A provided by the measuring head structure 2, and the outlet positions of the light guide L1, L2, L3 being coordinated in such a way that the distances of the outlet positions of the first and second light guide L1, 12, distinguish significantly from the outlet position of the third light guide L3, preferably by at least 0.4 mm.

The light guides L1, L2 and L3 are integrated in such a way in the measuring head structure 2 that they substantially enter perpendicularly from behind into the support surface A. The support surface A or the end windows of the light guide L1, L2, L3 can be equipped with a seal or a thin window structure so that the light guides are optically accessible, but not mechanically protected.

The distance of the outlet position of the first light guide L1 from the outlet position of the third light guide L3 is greater than the distance of the outlet position of the second light guide L2 from the outlet position of the third light guide L3. The distance of the outlet position of the first light guide L1 from the outlet position of the third light guide L3 corresponds roughly to the distance of the outlet position of the first light guide L1 from the outlet position of the second light guide L2.

The outlet positions of the light guide L1, L2, L3 form the vertices of a triangle, in which an interior angle defined between the legs extending towards the outlet position of the third light guide L3 is in the range from 79° to 94°, preferably 89°.

The distance of the outlet position of the first light guide L1 from the outlet position of the third light guide L3 in the concrete embodiment preferably amounts to 3.6 mm. The distance of the outlet position of the second light guide L2 from the outlet position of the third light guide L3 preferably amounts to 2.3 mm.

The light source device includes two separate LED light sources Q1, Q2 which each are associated to one of the light guides L1, L2. The light guides L1, L2 are iron-free multifilaments and integrated into a cladding with strain relief which is not shown here in detail. The spectrometer device includes a CCD array 7 by which the spectral distribution of the intensity of the light detected by the third light guide L3 can be determined by associating it to the wavelength.

The support surface is formed preferably as substantially circular or slightly elliptical area. The outlet positions of the light guides L1, L2, L3 are preferably determined in such a way that the centroid of a triangle defined accordingly by these outlet positions coincides substantially with the centroid of the support surface.

1. A measuring device with:
a light source device,
a spectrometer device, and
a measuring head structure,
wherein
the measuring head structure is coupled with the light source device over a first light guide and a second light guide as well as with the spectrometer device over a third light guide,
these light guides end in a support surface provided by the measuring head structure, and
the outlet positions of the light guide are coordinated in such a way that the distances of the outlet positions of the first and second light guide distinguish from the outlet position of the third light guide.

2. The measuring device according to claim 1, wherein the light guides substantially enter perpendicularly from behind into the support surface.

3. The measuring device according to claim 1, wherein the distance of the outlet position of the first light guide from the outlet position of the third light guide is greater than the distance of the outlet position of the second light guide from the outlet position of the third light guide.

4. The measuring device according to claim 1, wherein the distance of the outlet position of the first light guide from the outlet position of the third light guide corresponds to the distance of the outlet position of the first light guide from the outlet position of the second light guide.

5. The measuring device according to claim 1, wherein the outlet positions of the light guide represent the vertices of a triangle, and that an interior angle defined between the legs extending towards the outlet position of the third light guide is in the range from 79° to 94°.

6. The measuring device according to claim 1, wherein the distance of the outlet position of the first light guide from the outlet position of the third light guide amounts to 3.6 mm, the distance of the outlet position of the second light guide from the outlet position of the third light guide amounting to 2.3 mm.

7. The measuring device according to claim 1, wherein the light source device includes two separate LED light sources which each are associated to one of the light guides.

8. The measuring device according to claim 1, wherein the light guides are iron-free multifilaments.
